(19) United States
(12) Patent Application Publication
(10) Pub. No.: US 2021/0244842 A1
(43) Pub. Date: Aug. 12, 2021

(54) STERILIZATION CASSETTES FOR DENTAL AND BODY ART TOOLS

(71) Applicant: David S. Gillstrap, Bothell, WA (US)

(72) Inventor: David S. Gillstrap, Bothell, WA (US)

(21) Appl. No.: 17/174,376

(22) Filed: Feb. 12, 2021

Related U.S. Application Data
(60) Provisional application No. 62/975,717, filed on Feb. 12, 2020.

(51) Int. Cl. A61L 2/26; A61B 50/33; A61L 2/07

(52) U.S. Cl. CPC A61L 2/26; A61B 2050/3008; A61L 2/07; A61B 50/33

(57) ABSTRACT

Various aspects of a sterilization cassette system for the sterilization of body art instruments and dental instruments fashioned from material having improved sterilization and configured in various arrangements for improved storage efficiency. One or more trays comprising part of the sterilization cassette are configured and coupled to each other in various arrangements. Each of the one or more trays have a bottom side and four side walls which are disposed substantially perpendicular to the bottom side and the bottom side has a plurality of mutually sized and arranged holes which are configured such as to provided more efficient drainage.

### CROSS REFERENCE TO RELATED APPLICATION

This application claims benefit of U.S. Provisional Application No. 62/975717 filed on Feb. 12, 2020, which is incorporated by reference in its entirety.

### TECHNICAL FIELD OF THE INVENTION

The overall field of this invention is directed to cassettes for the sterilization of dental and body art instruments.

### BACKGROUND OF THE INVENTION

Instruments that are used in the medical, dental, and body art industries are generally sterilized prior to use. Instruments are generally sterilized using an autoclave. An autoclave may use steam and/or hot water to sterilize the instruments. Usually, the steam and/or water is injected into the autoclave for a period of time to sterilize. The instruments are placed in containers which are called sterilization cassettes. The cassettes are the containers used during sterilization and subsequent storage of the sterilized instruments.

Sterilization cassettes typically have a body defining one or more trays for holding the instruments. The trays typically have a closed position, in which the instruments are held within enclosed, porous compartments surrounded by and/or between the trays, and an open position, in which the instruments may be removed from the trays. A typical sterilization cassette will have a body defined by two opposing cassette trays connected in a clamshell configuration. Instrument retention members may be carried in the one or both trays. The two trays may be closed and secured together while retaining the instruments. Each tray may have a porous wall that allows the hot water and/or steam to enter into the compartment to sterilize the instruments therein while in an autoclave. The present cassettes have identified a need for improvements to the typical sterilization cassettes.

Various aspects of the present application include sterilization cassette systems which are configurable in various arrangements are shown, described, and/or claimed herein. The systems and members disclosed herein are preferably suitable for holding body art instrument as well as dental, and/or medical instruments during sterilization and subsequent storage.

Other aspects and advantages of the present disclosure will become apparent upon consideration of the following detailed description.

### BRIEF SUMMARY OF THE INVENTION

The invention as presently described is a system of connected trays that will be fashioned from medical/implant grade materials that can withstand high heat, as these mesh trays will be used in an autoclave to sterilize instruments. The non-limiting embodiments of a sterilization cassette described herein may include thermoplastic polymer, a medical/implant grade material that may be used to manufacture the mesh trays. The invention is comprised of various dimensions trays which may be molded into various shapes configured to hold a variety of instruments for sterilization and subsequent storage. The trays fashioned from such a medical/implant grade material have far better drainage than metal trays. This allows for faster drainage and thus faster drying time and also makes the sterilization process more efficient due to that property. The non-metal material also has the advantage that sensitive tools prone to getting scratched in metal trays or cassettes have a surface which is less likely to scratch the tools.

In the presently described disclosure, several embodiments of a sterilization cassette system are described. The sterilization cassette system comprises of a housing which may be made from a material including and not limited to Nylon 12, silicone, PTFE, metal, or metal with silicone overlay. The housing of the sterilization cassette may be described as having a length and a width which is comprised of one or more trays. Each tray of the sterilization cassette has a bottom side and four side walls which are disposed substantially perpendicular to the bottom side. In the non-limiting embodiments described herein, the bottom sides of the one or more trays include a plurality of holes which are arranged throughout the bottom side of each tray. The plurality of holes is mutually sized and spaced so as to allow even sterilization of the instruments that are placed in the tray by permitting steam and liquid to pass through into the one or more trays.

### BRIEF DESCRIPTION OF THE DRAWINGS

This description includes disclosure of the present system by way of exemplary, non-limiting embodiments illustrated in the accompanying drawings.

FIG. 1A illustrates a sterilization cassette in accordance with one aspect of the present invention.

FIG. 1B illustrates a sterilization cassette in a closed position in accordance with one aspect of the present invention.

FIG. 2A illustrates a sterilization cassette in a closed position in accordance with a second aspect of the present invention.

FIG. 2B illustrates a sterilization cassette in an open position in accordance with the second aspect of the present invention.

FIG. 2C illustrates a side view of a sterilization cassette in accordance with the second aspect of the present invention.

FIG. 3A illustrates a sterilization cassette in a closed position in accordance with third aspect of the present invention.

FIG. 3B illustrates a sterilization cassette in a closed position in accordance with the third aspect of the present invention.

FIG. 3C illustrates a side view of a sterilization cassette in accordance with the third aspect of the present invention.

### DETAILED DESCRIPTION OF THE INVENTION

In the Summary above, this Detailed Description, the claims below, and the accompanying drawings, reference is made to particular features of the invention. It is to be understood that the disclosure of the invention in this specification includes all possible combinations of such particular features. For example, where a particular feature is disclosed in the context of a particular aspect or embodiment of the invention, or a particular claim, that feature may also be used, to the extent possible, in combination with and/or in the context of other particular aspects and embodiments of the invention, and in the invention generally.

Where reference is made herein to a method comprising two or more defined steps, the defined steps may be carried out in any order or simultaneously (except where the context excludes that possibility), and the method may include one or more other steps which are carried out before any of the defined steps, between two of the defined steps, or after all the defined steps (except where the context excludes that possibility).

“Exemplary” is used herein to mean “serving as an example, instance, or illustration.” Any aspect described in this document as “exemplary” is not necessarily to be construed as preferred or advantageous over other aspects.

Throughout the drawings, like reference characters are used to designate like elements. As used herein, the term “coupled” or “coupling” may indicate a connection. The connection may be a direct or an indirect connection between one or more items. Further, the term “set” as used herein may denote one or more of any items, so a “set of items” may indicate the presence of only one item or may indicate more items. Thus, the term “set” may be equivalent to “one or more” as used herein.

It is the objective of the present invention to provide a sterilization cassette with an improved sterilization and storage efficiency. It is also the objective of the present invention to provide an instrument sterilization cassette which minimizes the chances of sensitive instruments from being scratched by being placed in the sterilization cassettes. It is also the object of the present invention to be able to hold a variety of instruments in a single cassette comprised of several trays coupled together. Still another objective is to provide a sterilization cassette with a housing fashioned from a material and having a plurality of mutually spaced openings in the cassette to optimize drainage during sterilization. Other objects will be apparent from the detailed description of the present invention.

In the non-limiting embodiments of a sterilization cassette described herein, the sterilization cassette is shown and described in several possible arrangements or configurations while retaining the advantage of the present invention. The non-limiting embodiments of the sterilization cassette are generally fashioned from a specific type of plastic, known as Nylon 12. Nylon 12 melts at a temperature of 175 degrees Celsius to 180 degrees Celsius and is suitable for the sterilization cassette as it can withstand the temperature specification of the sterilization process which is approximately 140 degrees Celsius. Other advantages of fashioning the sterilization cassette from Nylon 12 include and are not limited to good resistance to chemicals, low moisture absorption, good barrier properties, and good weathering resistance.

The non-limiting embodiments of the sterilization cassette described herein can also be fashioned from silicone which is typically resistant to chemical attack and insensitive to temperature changes. However, in order to add structural integrity to the sterilization cassettes being made from silicone, the sterilization cassettes would need to be fashioned from a harder material such as metal. The metal structure would adhere to the structural specifications of the sterilization cassette embodiments disclosed herein. Once the metal structure is fashioned into the sterilization cassettes, a silicone coating is applied to the structure. Other suitable materials that may be used to make the sterilization cassettes described herein include and are not limited to PTFE Teflon. It is also to be understood that the sterilization cassettes described herein may also be fashioned from metals that are generally used in sterilization of instruments.

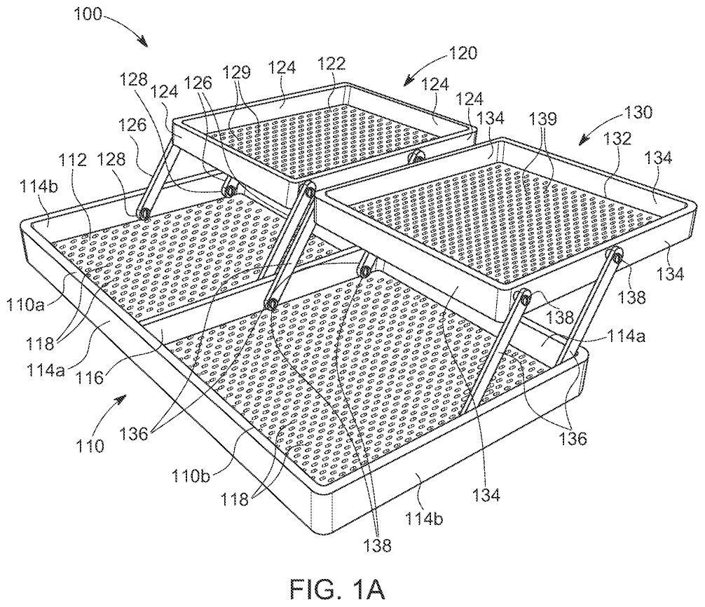

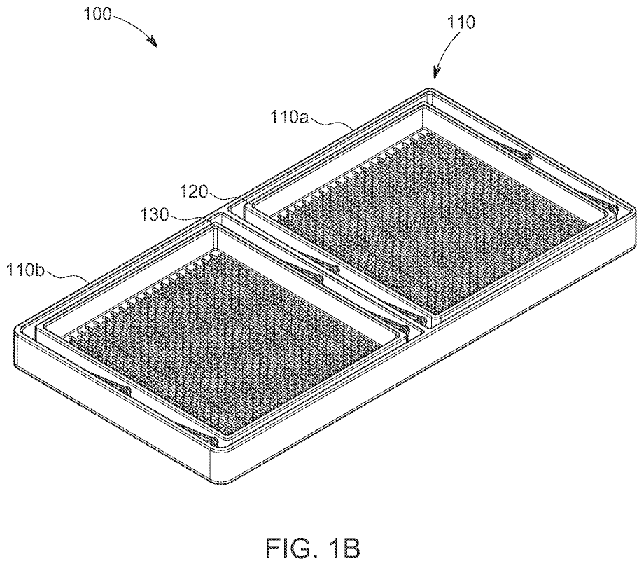

An instrument sterilization cassette in accordance with one aspect of the present invention is shown in FIGS. 1A and 1B for holding body art and/or dental instruments (not shown) during a sterilization process and for retaining the sterilized instruments post sterilization and during a procedure. In this embodiment, sterilization cassette 100 is shown to include a generally rectangular housing having a length and a width. The sterilization cassette 100 includes a bottom tray 110 and at least one inner tray. FIGS. 1A and 1B illustrate that the sterilization cassette 100 is comprised of two inner trays, a first inner tray 120 and a second inner tray 130. It is to be understood that the sterilization cassette 100 may also be comprised of one inner tray or more than one inner tray.

Referring to FIG. 1A, the bottom tray 110 has a body defined by a bottom side 112 and four side walls, wherein a pair of longer side walls 114a and a pair of shorter side walls 114b define the length and the width of the sterilization cassette 100. The pair of longer side walls 114a and the pair of shorter side walls 114b are substantially perpendicular to the bottom side 112. In this non-limiting embodiment, an opposing side to the bottom side 112 of the bottom tray 110 has an open arrangement wherein access to an interior compartment is not closed. (All relational directional terms used herein are used solely for convenience of description with respect to the drawings and are not otherwise intended as limiting the structural feature.) In an alternate embodiment (not shown), the bottom tray may comprise of a lid with a top side and four sides projecting downward, intended to be placed over the bottom tray opposite the bottom side 112. Alternatively, the lid may also be pivotally secured to the bottom tray by a hinge structure (not shown). Other attachment means are contemplated within the teaching of this invention.

Bottom tray 110 of the sterilization cassette 100 is also comprised of a divider wall 116 which is substantially perpendicular to the bottom side 112 parallel to the shorter side walls 114b. The divider wall 116 is integrated to the bottom side 110 and the longer side walls 114a such that the bottom tray is divided into two sections, a first section 110a and a second section 110b. As illustrated in FIG. 1A, the first section 110a and the second section 110b are generally equal in a length and a width, and therefore equal in size. It is also to be understood that the first section 110a and the second section 110b can be unequal in size wherein the divider wall 116 is integrated off center within the bottom tray. Further, it is also to be understood that the sterilization cassette 100 may be comprised of more than one divider wall 116, such that bottom side 112 may be divided into more than two sections. Additionally, it is also to be understood that the sterilization cassette 100 may not comprise of the divider wall 116 such that the bottom side 112 is not comprised of separate sections.

FIG. 1A also shows the sterilization cassette 100 comprising of two inner trays, the first inner tray 120 and the second inner tray 130. As discussed above, the sterilization cassette 100 may be comprised of at least one inner tray. Preferably, the sterilization cassette 100 may be comprised of an equal number of inner trays as the number of sections in the bottom tray 110. For example, in FIG. 1A and 1B, the sterilization cassette 100 is comprised of the bottom section 110 which includes two sections, the first section 110a and the second section 110b. Therefore, in this example, the sterilization cassette 100 is comprised of two inner trays, the first inner tray 120 and the second inner tray 130. As shown in FIG. 1B, the first inner tray 120 and the second inner tray 130 are sized to fit adjacent to each other within their corresponding section in the bottom tray 110, the first section 110a and the second section 110b, respectively. It is also to be understood that a lesser number of inner trays may also comprise part of the sterilization cassette, such that in the case of the above example, only one inner tray is provided and sized to fit within its corresponding section and one section does not receive a tray and has an open configuration.

First inner tray 120 and second inner tray 130 are each defined as having a body with a bottom side 122 and 132, respectively. The first inner tray 120 and second inner tray 130 also comprise of four side walls 124 and 134, respectively. The side walls 124 and 134 are generally defined as having a height shorter than a height of the bottom tray's 110 longer side wall 114a and shorter side wall 114a such that when the inner trays 120 and 130 are placed within the bottom tray 110, a top of the inner trays side walls 124 and 134 align with a top of the bottom tray's 110 longer side wall 114a and shorter side wall 114a. When the first inner tray 120 and the second inner tray 130 are located within the bottom tray 110, first and second inner tray 120 and 130 are not touching the bottom side 112 of the bottom tray and are intended to leave a space within the inner compartment of the bottom tray for including various instruments.

The first inner tray 120 and the second inner tray 130 are each individually connected to the bottom tray 110. As shown in FIG. 1A, first inner tray 120 is connected to the bottom tray 110 within the first section 110a with a set of four arms, referred to as first set of connecting arms 126. Each of the first set of connecting arms 126 has a first end and a second end. The first end of two of the first set of connecting arms 126 are connected to an inner side of the shorter side wall 114b on the first section 110a. The second end of these two first set of connecting arms are connected to the first inner tray 124 on an exterior side of the side wall 124 corresponding to the shorter side wall 114b. Additionally, the first end of the remaining two arms of the first set of connecting arms 126 are connected to the divider wall 116 by a pin 128 on a side facing the first section 110a. The second end of these two first set of connecting arms 126 are connected to the first inner tray 120 on an exterior side of the side wall 124 corresponding to the divider wall 116. Pins 128 connect each of the first arms 126 to the bottom tray 110 and the first inner tray 120 such that the first set of connecting arms 126 swivel at the pins 128 resulting in the first inner tray 120 being able to open up and away from the bottom tray 110 while simultaneously staying parallel to the bottom tray 110.

Similarly, second inner tray 130 is connected to the bottom tray 110 within the second section 110b with a set of four arms, referred to as second set of connecting arms 136, as shown in FIG. 1A. Each of the second set of connecting arms 136 has a first end and a second end. The first end of two arms of the second set of connecting arms 136 are connected to an inner side of the shorter side wall 114b on the second section 110b. The second end of these two second set of connecting arms 136 are connected to the second inner tray 130 on an exterior side of the side wall 134 corresponding to the shorter side wall 114b. Additionally, the first end of the remaining two arms of the second set of connecting arms 136 are connected to the divider wall 116 on a side facing the second section 110b. The second end of these two arms of the second set of connecting arms 136 are connected to the second inner tray 130 on an exterior side of the side wall 134 corresponding to the divider wall 116. Pins 138 connect each of the second set of connecting arms 136 to the bottom tray 110 and the second inner tray 130 such that the second set of connecting arms 136 swivel at the pins 138 resulting in the second inner tray 130 being able to open up and away from the bottom tray 110 while simultaneously staying parallel to the bottom tray 110. FIG. 1B shows first and second inner trays 120 and 130 in a closed position.

As illustrated in FIG. 1A, the bottom tray 110, the first inner tray 120, and the second inner tray 130 also comprise of a plurality of mutually sized and placed holes 118, 129, and 139, respectively. As shown, the bottom sides 112, 122, and 132 of each are arranged with the holes 118, 129, and 139, respectively, and which provide through-passages for liquid and/or steam to enter into the interior compartment of the bottom tray 110, the first inner tray 120 and the second inner tray 130. The holes 118, 129, and 139 also provide for proper drainage of any residual liquid. Each of the holes 118, 129, and 139 are fashioned with beveled edges (not shown) providing for superior drainage such that during a drying phase the liquid flows down the beveled edges and prevents any pooling of liquid around the instrument(s) placed within the sterilization cassette 100. The holes 118, 129, and 139 may be formed in any number of different shapes.

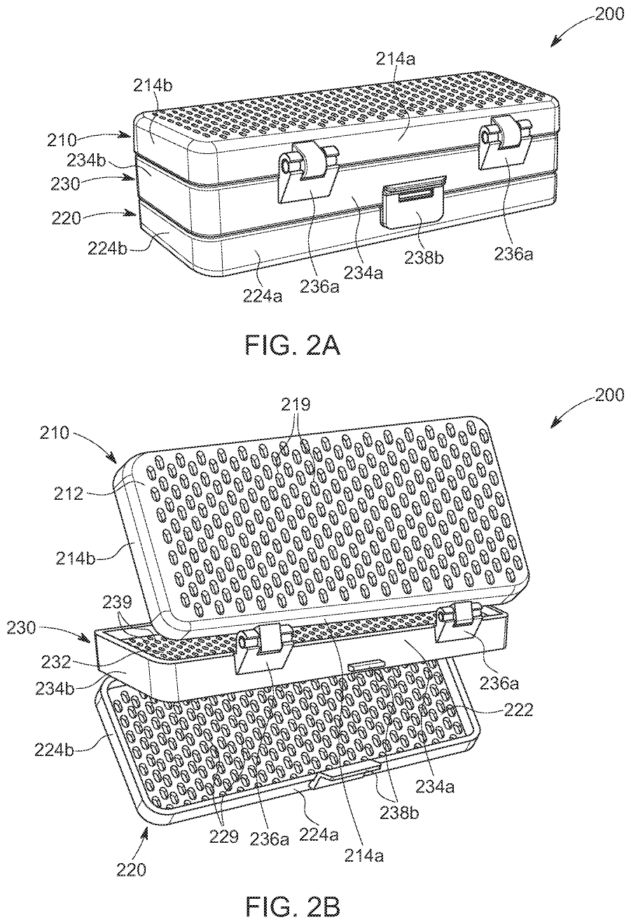

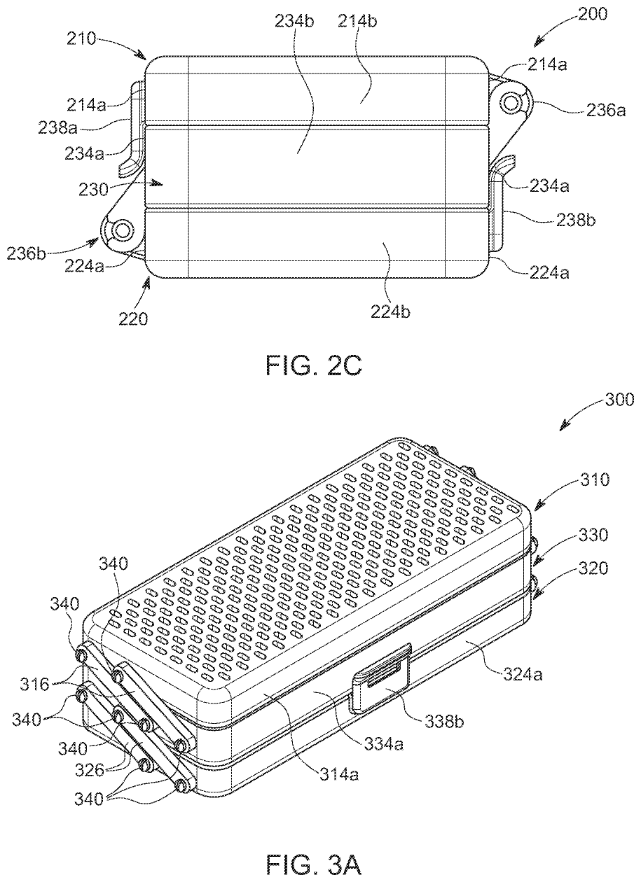

An instrument sterilization cassette in accordance with a second aspect of the present invention is shown in FIGS. 2A to 2C for holding body art and/or dental instruments (not shown) during a sterilization process and for retaining the sterilized instruments post sterilization and during a procedure. In this embodiment, sterilization cassette 200 is shown to be comprised of three trays, a first outer tray 210, a second outer tray 220, and an inner tray 230 which are connected to each other, either directly or indirectly, and generally define an enclosed housing with a length and a width. As shown in the figures, each of the trays 210, 220, 230 has a generally rectangular configuration, however other configurations are also intended to be encompassed in this disclosure.

FIG. 2A shows the sterilization cassette 200 in a closed position. As illustrated, the first outer tray 210 and the second outer tray 220 are indirectly connected to each other via the inner tray 230 which is arranged in between and secured to the first outer tray 210 and the second outer tray 220. In the illustration, for relational purposes only, the first outer tray 210 is shown on top and the second outer tray 220 is shown on the bottom, however, the configuration of the sterilization cassette 200 allows the cassette to be turned around wherein the second outer tray 220 can be on the top and still maintain the same arrangement.

FIG. 2B shows the sterilization cassette in an open position, wherein the first outer tray 210 and the second outer tray 220 are connected to the inner tray 230 in a clamshell arrangement, wherein an interior compartment is formed between each. The interior compartments between each arrangement are sized and shaped to receive one or more instruments, such as are used in the body art industry and dental industry, and completely enclose the instruments. As can be seen in FIG. 2B, in the open position the interior compartments are exposed allowing the insertion or removal of instruments. The sterilization cassette 200 is adapted such that both the first outer tray 210 and the second outer tray 220 can be selectively opened when the opposing outer tray is placed on a surface. For example, the sterilization cassette 200 may be placed on a surface with the first outer tray 210 on top, wherein the first outer tray 210 may be opened to expose the interior compartment. Conversely, the second outer tray 220 may be on top allowing access to the interior compartment in an open position.

First outer tray 210 and second outer tray 220 are each defined as having a body with a bottom side 212 and 222, respectively. The first outer tray 210 and second outer tray 220 also comprise of four side walls each, wherein each having a pair of longer side walls 214a, 224a and a pair of shorter side walls 214b, 224b, respectively, which are substantially parallel to their bottom sides 212, 222, respectively. The side walls 214a, 214b, 224a, 224b define the length and the width of the sterilization cassette 200. The inner tray 230 is also defined as having a body with a base 232 and four side walls wherein a pair of longer side walls 234a and a pair of shorter side walls 234b, also define the length and width of the sterilization cassette 200 and are substantially parallel to the base 232. The base 232 of the inner tray 230 is generally placed in a middle plane of the side walls 234a and 234b, such that the side walls 234a, 234b depend equally upward and downward from and surround the base 232. Together, the first outer tray 210, the second outer tray 220, and the inner tray 230 provide an enclosed housing with two enclosed interior compartments for the placement of instruments.

As discussed above, the first outer tray 210 and the second outer tray 220 are both connected to the inner tray 230, wherein the inner tray 230 is arranged in the middle with the first outer tray 210 on one side and the second outer tray 220 on a second side. The first outer tray 210 and the second outer tray 220 are each connected to the inner tray 230 by any means that allows the outer trays 210, 220 to open in a clamshell manner as shown in FIG. 2B. As shown in FIGS. 2A to 2C the connection means may include a hinge 236. FIGS. 2A and 2B show an angled front perspective view of the sterilization cassette 200 with two hinges, such as first hinges 236a integrated onto the longer side walls 214a, 234a of the first outer tray 210 and the inner tray 230, respectively. The first hinges 236a connect the first outer tray 210 and the inner tray 230 at one longer side wall 214a, 234a that are vertically aligned for each of these trays 210, 230. In this embodiment and shown in FIG. 2C, a similar arrangement is integrated onto the second outer tray 220, wherein the opposing longer side walls 224a, 234a which are vertically aligned with each other are integrated with two hinges, such as second hinges 236b. It is to be understood that each opposing side wall may be arranged with one hinge or more than one hinge.

Releasable securing means also comprise part of the sterilization cassette 200. A first releasable securing means is integrated on each of the longer side walls 234a of the inner tray 230 and on opposing vertically aligned longer side walls 214a, 224a of the first outer tray 210 and the second outer tray 220, respectively. As best seen in FIG. 2C, a first releasable securing means 238a is integrated on one of the longer side walls 234a of the inner tray 230 and the vertically aligned longer side wall 214a of the first outer tray 210. The first releasable securing means 238a engages and releasably secures the first outer tray 210 and the inner tray 230 together. A second releasable securing means 238b is integrated onto the opposing longer side wall 234a of the inner tray 230 and the vertically aligned longer side wall 224a of the second outer tray 220. The second releasable securing means 238b engages and releasably secures the second outer tray 220 and the inner tray 230 together. As best seen in FIG. 2C, first and second releasable securing means 238a, 238b may include and not be limited to a snap type closure means.

As best seen in FIG. 2B, the first outer tray 210, the second outer tray 220, and the inner tray 230 also comprise of a plurality of mutually sized and placed holes 219, 229, and 239, respectively. As shown, the bottom sides 212, 222, of first and second outer tray 210, 220, respectively, are each arranged with plurality of holes 219, 229. Also, the base 232 of the inner tray 230 is arranged with the holes 239. The holes 219, 229, 239 provide through-passages for liquid and/or steam to enter into the interior compartments of the first outer tray 210, the second outer tray 220, and the inner tray 230. The holes 219, 229, and 239 also provide for proper drainage of any residual liquid. Each of the holes 219, 229, and 239 are fashioned with beveled edges (not shown) providing for superior drainage such that during a drying phase the liquid flows down the beveled edges and prevents any pooling of liquid around the instrument(s) placed within the sterilization cassette 200. The holes 219, 229, and 239 may be formed in any number of different shapes.

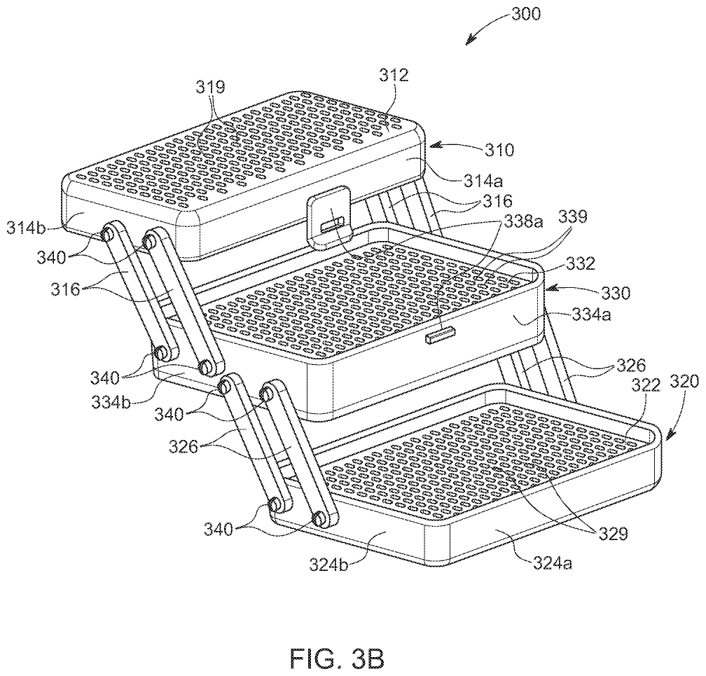

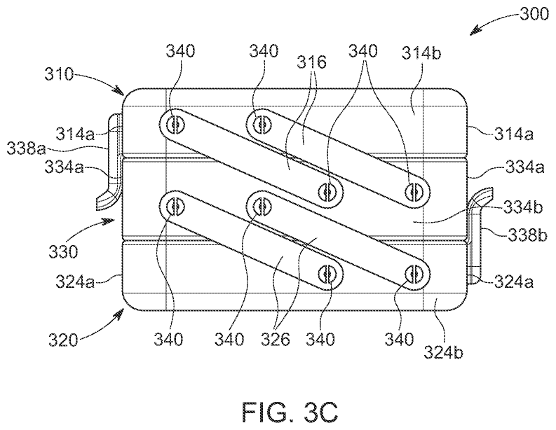

An instrument sterilization cassette in accordance with a third aspect of the present invention is shown in FIGS. 3A to 3C for holding body art and/or dental instruments (not shown) during a sterilization process and for retaining the sterilized instruments post sterilization and during a procedure. In this embodiment, sterilization cassette 300 is shown to be comprised of three trays, a first outer tray 310, a second outer tray 320, and an inner tray 330 which are connected to each other, either directly or indirectly, and generally define an enclosed housing with a length and a width. As shown in the figures, each of the trays 310, 320, 330 has a generally rectangular configuration, however other configurations are also intended to be encompassed in this disclosure.

FIG. 3B shows the sterilization cassette 300 in an open position, wherein the first outer tray 310 and the second outer tray 320 are each connected to the inner tray 330, wherein an interior compartment is formed between each. The trays 310, 320, and 330 have a tiered arrangement when in the open position. The interior compartments between each arrangement are sized and shaped to receive one or more instruments, such as used in the body art industry and dental industry, and completely enclose the instruments. As can be seen in FIG. 3B, in the open position the interior compartments are exposed allowing the insertion or removal of instruments. The sterilization cassette 300 is adapted such that both the first outer tray 310 and the second outer tray 320 can be selectively opened regardless of which outer tray 310, 320 is on the bottom and placed on a surface. For example, the sterilization cassette 300 may be placed on a surface with the second outer tray 310 on bottom. In this example, the first outer tray 310 may be opened to expose the interior compartment, the bottom tray 320 may be opened, or both trays 310, 320 may be opened. Conversely, if the first outer tray 310 is on the bottom, the same opening arrangement allowing access to the interior compartments is still possible.

Similar to the second embodiment (See, FIGS. 2A to 2C, sterilization cassette 200), the sterilization cassettes 300 first outer tray 310 and second outer tray 320 are each defined as having a body with a bottom side 312 and 322, respectively. The first outer tray 310 and the second outer tray 320 also comprise of four side walls each, wherein each having a pair of longer side walls 314a, 324a and a pair of shorter side walls 314b, 324b, respectively, and define the length and the width of the sterilization cassette 300. The inner tray 330 is also defined as having a body with a base 332 and four side walls wherein a pair of longer side walls 334a and a pair of shorter side walls 334b, also define the length and width of the sterilization cassette 300. The base 332 of the inner tray 330 is generally placed in a middle plane of the side walls 334a and 334b, such that the side walls 334a and 334b depend equally upward and downward from and surround the base 332. Together, the first outer tray 310, second outer tray 320, and the inner tray 330 provide two enclosed interior compartments for the placement of instruments.

The first outer tray 310 and the second outer tray 320 are both connected to the inner tray 330, wherein the inner tray 330 is arranged in the middle with the first outer tray 310 on one side and the second outer tray 320 on a second side. The first outer tray 310 and the second outer tray 320 are each connected to the inner tray 330 by any means that allows the trays 310, 320, 330 to open in the tiered arrangement. As best seen in FIGS. 3B and 3C, the inner tray 330 is connected to the first outer tray 310 by a first set of connecting arms 316, with two arms on each side. Similarly, the inner tray 330 is connected to the second outer tray 320 by a second set of connecting arms 326, with two arms on each side. Each of the first set of connecting arms 316 and the second set of connecting arms 326 has a first end and a second end. The first ends of the first set of connecting arms 316 are connected to an outer sides of the shorter side walls 314b of the first outer tray 310, with two arms of the first set of connecting arms 316 on each of the shorter side walls 314b. Similarly, the first ends of the second set of connecting arms 326 are connected to an outer sides of the shorter side walls 324b of the second outer tray 320, with two arms of the second set of connecting arms 326 on each of the shorter side walls 324b. The second ends of first set and second set of connecting arms 316, 326 are connected to an outer side of the shorter side walls 334b of the inner tray 330, wherein the second sides of first set and second set of connecting arms 316, 326 are in line along a length of the shorter side walls 334b. Pins 340 connect each of the first set of connecting arms 316 and the second set of connecting arms 326 to their respective trays resulting in the arms 316, 326 being able to swivel at the pins 340. FIG. 3B illustrates that the first set of connecting arms 316 and the second set of connecting arms 326 swiveling at the pins 340 results in the first outer tray 310 being able to open up and away from the inner tray 330 while the inner tray 330 is also able to open up and away from the second outer tray 320.

Similar to the embodiment in FIGS. 2A to 2C, the sterilization cassette 300 of the present embodiment shown in FIGS. 3A to 3C, also comprises a releasable securing means. A releasable securing means is integrated on each of the longer side walls 334a of the inner tray 330 and on the opposing longer side walls 314a, 324a of the first outer tray 310 and the second outer tray 320, respectively. As best seen in FIG. 3C, a first releasable securing means 338a is integrated on one of the longer side walls 334a of the inner tray 330 and the vertically aligned longer side wall 314a of the first outer tray 310. The first releasable securing means 338a engages and releasably secures the first outer tray 310 and the inner tray 330 together. As shown in the figures, a second releasable securing means 338b is integrated onto the opposing longer side wall 334a of the inner tray 330 and the vertically aligned longer side wall 324a of the second outer tray 320. The second releasable securing means 338b engages and releasably secures the second outer tray 320 and the inner tray 330 together. As best seen in FIG. 3C, first and second releasable securing means 338a, 338b, may include and not be limited to a snap type closure means. It is also to be understood that alternate embodiments can include the first releasable securing means 338a and the second securing means 338b on the same side.

The first outer tray 310, the second outer tray 320, and the inner tray 330 of the sterilization cassette 300 also comprise of a plurality of mutually sized and placed holes 319, 329, and 339, respectively. As best shown in FIG. 3B, the bottom sides 312, 322, of first and second outer tray 310, 320, respectively, are each arranged with the plurality of holes 319, 329. Also, the base 332 of the inner tray 330 is arranged with the holes 339. The holes 319, 329, 339 provide through-passages for liquid and/or steam to enter into the interior compartments of the first outer tray 310, the second outer tray 320, and the inner tray 330, and also provide for proper drainage of any residual liquid. Each of the holes 319, 329, and 339 are fashioned with beveled edges (not shown) providing for superior drainage such that during a drying phase the liquid flows down the beveled edges and prevents any pooling of liquid around the instrument(s) placed within the sterilization cassette 300. The holes 319, 329, and 339 may be formed in any number of different shapes.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description but is not intended to be exhaustive or limited to the invention in the form disclosed. Many modifications and variations will be apparent to those of ordinary skill in the art without departing from the scope and spirit of the invention. The embodiments were chosen and described in order to best explain the principles of the invention and the practical application, and to enable others of ordinary skill in the art to understand the invention for various embodiments with various modifications as are suited to the particular use contemplated. The present invention according to one or more embodiments described in the present description may be practiced with modification and alteration within the spirit and scope of the appended claims. Thus, the description is to be regarded as illustrative instead of restrictive of the present invention.

What may be claimed:

1. A sterilization cassette system, comprising:
a housing, having a length and a width, comprising:
one or more trays, wherein each of the one or more trays having a bottom side and four side walls disposed substantially perpendicular to the bottom side;
a plurality of holes, mutually sized and arranged throughout the bottom side of each of the one or more trays; and
wherein a material to make the housing is selected from Nylon 12, silicone, PTFE, metal, or metal overlaid with silicone.

2. The sterilization cassette system of claim 1, wherein the housing comprises of a bottom tray, a first inner tray, and a second inner tray, wherein the bottom tray defines the length and the width of the housing, and
the first inner tray and the second inner tray having a length and a width sized smaller than the length and the width of the housing such that the first inner tray and second inner tray fit adjacently within the bottom tray.

3. The sterilization cassette system of claim 2, wherein the bottom tray comprises of a divider wall which is integrated within the bottom tray and is substantially perpendicular to the bottom tray and extends from a longer side wall to an opposing longer side wall of the bottom tray forming a first section and a second section with the bottom tray.

4. The sterilization cassette system of claim 3, wherein the first inner tray fits within the first section of the bottom tray and one or more arms connect the second inner tray to the bottom tray;
wherein the first inner tray sits suspended above the bottom side of the first section to form a space between a bottom side of the first inner tray and the bottom side of the first section;
wherein the second inner tray fits within the second section of the bottom tray and one or more arms connect the second inner tray to the bottom tray; and
wherein the second inner tray sits suspended above the bottom side of the second section to form a space between a bottom side of the second inner tray and the bottom side of the second section.

5. The sterilization cassette system of claim 4, wherein a first set of arms comprising at least two arms connect the first inner tray to the bottom tray, wherein at least one arm of the first set of arms connects a shorter side wall of the bottom tray to a corresponding side wall of the first inner tray and at least a second arm of the first set of arms connects the divider wall of the bottom tray to a corresponding side wall of the first inner tray;
wherein a plurality of pins connect each of the arms of the first set of arms at the shorter side wall, the divider wall, and the corresponding side walls of the first inner tray; and
each of the first set of arms swivel at each of the pins of the plurality of pins such that the first inner tray can be moved up and away and back to sit within the first section.

6. The sterilization cassette system of claim 4, wherein a second set of arms comprising at least two arms connect the second inner tray to the bottom tray, wherein at least one arm of the second set of arms connects a shorter side wall of the bottom tray to a corresponding side wall of the second inner tray and at least a second arm of the second set of arms connects the divider wall of the bottom tray to a corresponding side wall of the second inner tray;
wherein a plurality of pins connects each of the arms of the second set of arms at the shorter side wall, the divider wall, and the corresponding side walls of the second inner tray; and
each of the arms of the second set of arms swivel at each of the pins of the plurality of pins such that the second inner tray can be moved up and away and back to sit within the second section.

7. The sterilization cassette system of claim 1, wherein the plurality of holes is each configured with a beveled edge.

8. A sterilization cassette system, comprising:
a housing, having a length and a width, comprising:
a first outer tray and a second outer tray, wherein the first outer tray and the second outer tray each having a bottom side and four side walls disposed substantially perpendicular to the bottom side;
an inner tray, having a base and four side walls disposed substantially perpendicular to the base and extending on each side of the base;
a plurality of holes, mutually sized and arranged throughout the bottom side of each of the first outer tray and the second outer tray and the base of the inner tray; and
wherein a material to make the housing is selected from Nylon 12, silicone, PTFE, metal, or metal overlaid with silicone.

9. The sterilization cassette system of claim 8, wherein the first outer tray, the second outer tray, and the inner tray define the length and the width of the housing, and
wherein the first outer tray is connected to the inner tray on a side and the second outer tray is connected to the inner tray on another side such that the inner tray is arranged between the first outer tray and the second outer tray, and
the side walls of the first outer tray, the second outer tray, and the inner tray are vertically aligned to form an enclosed housing.

10. The sterilization cassette system of claim 9, wherein a first hinge connects the first outer tray to the inner tray, wherein the first hinge is configured on a longer side wall of the inner tray and a vertically aligned longer side wall of the first outer tray; and
wherein a second hinge connects the second outer tray to the inner tray, wherein the second hinge is configured on an opposing longer side wall of the inner tray and a vertically aligned longer side wall of the second outer tray.

11. The sterilization cassette system of claim 9, wherein a first set of arms comprising at least two arms connect the first outer tray to the inner tray;
wherein at least one arm of the first set of arms connects a shorter side wall of the first outer tray to a vertically aligned shorter side wall of the inner tray and at least a second arm of the first set of arms connects an opposing shorter side wall of the first outer tray to a vertically aligned shorter side wall of the inner tray;
wherein a second set of arms comprising at least two arms connect the second outer tray to the inner tray; and
wherein at least one arm of the second set of arms connects a shorter side wall of the second outer tray to a vertically aligned shorter side wall of the inner tray and at least a second arm of the second set of arms connects an opposing shorter side wall of the second outer tray to a vertically aligned shorter side wall of the inner tray.

12. The sterilization cassette system of claim 11, wherein a plurality of pins connects the first set of arms to the first outer tray and to the inner tray, wherein each of the first set of arms connecting the first outer tray and the inner tray swivel at each of the pins of the plurality of pins such that the first outer tray can be moved up and away from the inner tray and back to align with the inner tray; and
wherein a plurality of pins connects the second set of arms to the second outer tray and to the inner tray, wherein each of the arms of the second set of arms connecting the second outer tray and the inner tray swivel at each of the pins of the plurality of pins such that the second outer tray can be moved up and away from the inner tray and back to align with the inner tray.

13. The sterilization cassette system of claim 9, wherein the housing comprises of a releasable securing means, wherein a first releasable securing means is integrated on a longer side wall of the inner tray and a vertically aligned longer side wall of the first outer tray such that the first outer tray and the inner tray are releasably secured together;
wherein a second releasable securing means is integrated on the same or an opposing longer side wall of the inner tray and a vertically aligned longer side wall of the second outer tray such that the second outer tray and the inner tray are releasably secured together; and
wherein the first outer tray and the second outer can be opened individually.

14. The sterilization cassette of claim 8, wherein the plurality of holes is each configured with a beveled edge.

15. A sterilization cassette system, comprising:
a housing, having a length and a width, comprising:
a first outer tray and a second outer tray, wherein the first outer tray and the second outer tray each having a bottom side and four side walls disposed substantially perpendicular to the bottom side;
an inner tray, having a base and four side walls disposed substantially perpendicular to the base and extending on each side of the base;
wherein the first outer tray is connected to the inner tray on a side and the second outer tray is connected to the inner tray on another side such that the inner tray is arranged between the first outer tray and the second outer tray, and the side walls of the first outer tray, the second outer tray, and the inner tray are vertically aligned to form an enclosed housing;
a plurality of holes, mutually sized and arranged throughout the bottom side of each of the first outer tray and the second outer tray and the base of the inner tray; and
wherein a material to make the housing is selected from Nylon 12, silicone, PTFE, metal, or metal overlaid with silicone.

16. The sterilization cassette system of claim 15, wherein a first hinge connects the first outer tray to the inner tray, wherein the first hinge is configured on a longer side wall of the inner tray and a vertically aligned longer side wall of the first outer tray; and
wherein a second hinge connects the second outer tray to the inner tray, wherein the second hinge is configured on an opposing longer side wall of the inner tray and a vertically aligned longer side wall of the second outer tray.

17. The sterilization cassette system of claim 15, wherein a first set of arms comprising at least two arms connect the first outer tray to the inner tray;
wherein at least one arm of the first set of arms connects a shorter side wall of the first outer tray to a vertically aligned shorter side wall of the inner tray and at least a second arm of the first set of arms connects an opposing shorter side wall of the first outer tray to a vertically aligned shorter side wall of the inner tray;
wherein a second set of arms comprising at least two arms connect the second outer tray to the inner tray; and
wherein at least one arm of the second set of arms connects a shorter side wall of the second outer tray to a vertically aligned shorter side wall of the inner tray and at least a second arm of the second set of arms connects an opposing shorter side wall of the second outer tray to a vertically aligned shorter side wall of the inner tray.

18. The sterilization cassette system of claim 17, wherein a plurality of pins connects the first set of arms to the first outer tray and to the inner tray, wherein each of the arms of the first set of arms connecting the first outer tray and the inner tray swivel at each of the pins of the plurality of pins such that the first outer tray can be moved up and away from the inner tray and back to align with the inner tray; and
wherein a plurality of pins connects the second set of arms to the second outer tray and to the inner tray, wherein each of the arms of the second set of arms connecting the second outer tray and the inner tray swivel at each of the pins of the plurality of pins such that the second outer tray can be moved up and away from the inner tray and back to align with the inner tray.

19. The sterilization cassette system of claim 15, wherein the housing comprises of a releasable securing means, wherein a first releasable securing means is integrated on a longer side wall of the inner tray and a vertically aligned longer side wall of the first outer tray such that the first outer tray and the inner tray are releasably secured together;
wherein a second releasable securing means is integrated on the same or an opposing longer side wall of the inner tray and a vertically aligned longer side wall of the second outer tray such that the second outer tray and the inner tray are releasably secured together; and
wherein the first outer tray and the second outer can be opened individually.

20. The sterilization cassette system of claim 15, wherein the plurality of holes is each configured with a beveled edge.
